SEQUENCE LISTING (8 sequences)

SEQ ID NO: 1 - DNA, 30 bp, synthetic construct
  source 1..30: mol_type = other DNA
aaattaaccaaggaggtgaaaggtagcatg

SEQ ID NO: 2 - DNA, 30 bp, synthetic construct
  source 1..30: mol_type = other DNA
tgtatattagaaaggaggaatatataaatg

SEQ ID NO: 3 - protein, 24 aa, synthetic construct
MKKTMITFSLVLMSLFGVASGASA

SEQ ID NO: 4 - protein, 25 aa, synthetic construct
MYIKKCIGSILFLLLFCSSALPAKA

SEQ ID NO: 5 - protein, 35 aa, synthetic construct
GPGQQAAAAAGPGQQGPGQQGPGQQGPGEQGPGSG

SEQ ID NO: 6 - protein, 169 aa, synthetic construct
MKKTMITFSLVLMSLFGVASGASAATNTYGPGQQAAAAAGPGQQGPGQQGPGQQGPGEQGPGSGGPGQQAAAAAGPGQQGPGQQGPGQQGPGEQGPGSGGPGQQAAAAAGPGQQGPGQQGPGQQGPGEQGPGSGGPGQQAAAAAGPGQQGPGQQGPGQQGPGEQGPGSG

SEQ ID NO: 7 - protein, 170 aa, synthetic construct
MYIKKCIGSILFLLLFCSSALPAKADVPTTGPGQQAAAAAGPGQQGPGQQGPGQQGPGEQGPGSGGPGQQAAAAAGPGQQGPGQQGPGQQGPGEQGPGSGGPGQQAAAAAGPGQQGPGQQGPGQQGPGEQGPGSGGPGQQAAAAAGPGQQGPGQQGPGQQGPGEQGPGSG

SEQ ID NO: 8 - protein, 146 aa, synthetic construct
GPGQQAAAAAGPGQQGPGQQGPGQQGPGEQGPGSGGPGQQAAAAAGPGQQGPGQQGPGQQGPGEQGPGSGGPGQQAAAAAGPGQQGPGQQGPGQQGPGEQGPGSGGPGQQAAAAAGPGQQGPGQQGPGQQGPGEQGPGSGHHHHHH

(19) United States
(12) Patent Application Publication
(10) Pub. No.: US 2026/0356897 A1
(43) Pub. Date: Nov. 12, 2026

(54) RECOMBINANT PRODUCTION OF SILK PROTEINS VIA BACILLUS MEGATERIUM SECRETION

(71) Applicant: Rensselaer Polytechnic Institute, Troy, NY (US)

(72) Inventors: ALEXANDER JOSEPH CONNOR, Slingerlands, NY (US); MATTHEOS AG KOFFAS, Niskayuna, NY (US); RUNYE HELEN ZHA, Troy, NY (US)

(73) Assignee: Rensselaer Polytechnic Institute, Troy, NY (US)

(21) Appl. No.: 19/481,052

(22) PCT Filed: May 3, 2024
(86) PCT No.: PCT/US2024/027669

§ 371 Date: Nov. 3, 2025

Related U.S. Application Data
(60) Provisional application No. 63/463,733, filed on May 3, 2023.

(51) Int. Cl. C07K 14/435; C07K 14/32; C12N 15/75

(52) U.S. Cl. CPC C07K 14/43518; C07K 14/32; C12N 15/75

(57) ABSTRACT

Gram-positive Bacillus sp. bacteria, specifically B. megaterium, is modified with recombinant genes to facilitate production of disordered proteins, such as recombinant silk proteins. Recombinant genes are inserted into pT7 vectors which are transformed into B. megaterium for expression. The genes include one or more translational initiation sites (TIS), signal sequences (SS), and gene fragments encoding the silk proteins, e.g., A5 4mer spidroins. The TIS can include the vector's original site, or a recombinant one. The SS encode signal peptides such as Yoch or Yngk. The recombinant B. megaterium is grown and expressed in a minimal medium supplemented by a plurality of amino acids and using glucose as a carbon source. The modified B. megaterium generate the protein product and further secrete that product into the growth media, mitigating toxicity of the disordered proteins and facilitating continuous thereof. Previous recombinant silk protein production methods suffered from low-yields and batch-production limitations.

### CROSS-REFERENCE TO RELATED APPLICATIONS

This application claims the benefit of U.S. Provisional Patent Application No. 63/463,733, filed May 3, 2023, which is incorporated by reference as if disclosed herein in its entirety.

### STATEMENT REGARDING FEDERALLY SPONSORED RESEARCH AND DEVELOPMENT

This invention was made with U.S. Government support under Grant Number 2036768 awarded by the National Science Foundation. The United States Government has certain rights in the invention.

### BACKGROUND

Silk proteins are of intense interest to engineers and researchers due to their unparalleled combination of chemical and physical properties, varied morphologies, and diverse applications. Two of the most extensively studied silk materials include the cocoon silk of domesticated Bombyx mori silkworms and dragline silk from orb-weaving spiders such as A. diadematus. Dragline silk is used by orb-weaving spiders to form the sturdy framework of their webs and possesses an advantageous combination of tensile strength and extensibility, imparting it with a toughness that surpasses numerous high-performance man-made and natural materials.

The properties of spidroins, which are proteins found in spider silk, arise from specific peptide motifs, chemical interactions, and hierarchical organization that tend to be conserved among various spider species. The spinning glands of silk-producing species generally assemble individual spidroins into nano-scale bundles called fibrils. These fibrils then assemble to form a mature, micro-scale silk fiber with naturally occurring fibers also containing a coating that is comprised of lipids and glycoproteins. Dragline spidroins are generally quite large, often several hundred kilodaltons in size. These proteins take the form of linear segmented copolymers, with small non-repetitive N and C-terminal domains (approximately 100-150 amino acids) flanking a large repetitive core domain. The core domain generally includes two different segments which vary in their amino acid content and function, with one segment contributing to extensibility and the other to tensile strength and stiffness. The segments of spidroins responsible for imparting tensile strength and stiffness aggregate to form beta-sheet crystals and are rich in alanine residues. The segments responsible for spider silk's extensibility contain a more diverse set of residues and form an amorphous matrix with a relatively lower beta-sheet content. It is the interplay between these two segments that imparts spider silk with a remarkably high toughness and strength-to-weight ratio.

Referring now to FIG. 1, the repetitive region of dragline spidroins can comprise approximately 90% of the total protein. Generally, dragline fibers from spiders are made up of two types of spidroin proteins, major ampullate 1 (MaSp1) and major ampullate 2 (MaSp2). The repetitive domain of MaSp1 is characterized by tandem alanine repeats (Aₙ where n is approximately 6-9) that alternate with a glycine-rich region characterized by GGX motifs, where X often represents tyrosine (Y), glutamine (Q), or leucine (L). MaSp2 also contains tandem alanine repeats, but its glycine rich region is also high in proline (P) and is characterized by GPGXX and GGX, motifs where X often represents Q, Y, L, G, or serine(S). In both MaSp1 and MaSp2, the tandem alanine regions are responsible for the formation of beta-sheets and beta-sheet crystals. Likewise, the glycine-rich regions are responsible for the formation of an amorphous matrix that can contain secondary structures including alpha helices, beta-turns, and random coils. Orb-weaving spiders produce several other types of silk other than dragline, such as flagelliform and minor ampullate silk (MiSp). Flagelliform silk is markedly different from dragline and contains only flexible regions dominated by GPGXX and GGX motifs. Minor ampullate silk contains (GA)ₙ, (A)ₙ, and GGX motifs. In contrast to dragline silk, flagelliform and minor ampullate silk both contain non-repetitive spacers that are rich in serine.

Silk proteins from other species, such as that of the silkworm Bombyx mori and the bagworm E. variegata, present substantially different primary sequence composition compared to dragline spidroins. However, the basic organization of the proteins into crystalline and amorphous domains as well as the use of segments rich in glycine and alanine remains conserved. The main protein in Bombyx mori silk protein (silk fibroin) contains a repetitive core with 12 crystalline segments that are rich in GAGAGX peptide motifs, where X is often S or Y. The crystalline segments are connected by short and flexible linkers that are highly conserved, non-repetitive, and contain a higher diversity of amino acids than spidroin. Silk from the bagworm E. variegata exemplifies additional iterations produced through the evolution of silk proteins. Its crystalline region combines motifs from both Bombyx mori and orb-weaving spiders and is characterized by adjacent tandem alanine and (GA), repeats. In partial similarity to dragline spidroins, the flexible region is dominated by GGY and GSG motifs.

Recombinant spidroins have been produced in a diverse set of host organisms, including bacteria, yeast, mammalian cells, insect cells, transgenic plants, and transgenic animals. However, efforts to produce recombinant spidroins have suffered from low titers, preventing the production and utilization of artificial spider silk at a commercial scale. Additionally, expressing recombinant spidroins in bacteria is often plagued by a high degree of plasmid instability, inclusion body formation, low solubility of the spidroin constructs, and transcriptional and translational errors. Attempts to express recombinant silk in eukaryotic cells or transgenic animals generally suffers from these same challenges.

Commercial production of spidroins by engineered bacteria such as E. coli has been hindered predominantly by two issues. First, there is evidence that host systems experience metabolic stress in response to producing these constructs in minimal media. The unusually high glutamine and proline content of silk protein depletes the host strain of ATP and NADPH, leading to suboptimal titers. Secondly, the observable product toxicity of a recombinant silk construct (which is likely due to a high level of intrinsic disorder) contributes to suboptimal titers by increasing oxidative and acetate stress responses within the host, initiating feedback loops within the TCA cycle and ED pathway that lower available ATP. This feedback loop is strengthened by the observation that glutamine and proline are TCA cycle amino acids.

### SUMMARY

Aspects of the present disclosure are directed to a method of making recombinant silk protein. In some embodiments, the method includes inserting one or more recombinant genes into a plurality of Bacillus sp. bacteria to form recombinant Bacillus sp. bacteria. In some embodiments, the plurality of Bacillus sp. bacteria includes B. megaterium bacteria. In some embodiments, the recombinant genes include one or more translational initiation sites (TIS), one or more polynucleotides encoding one or more signal peptides (SS), and one or more gene fragments encoding silk proteins (SP). In some embodiments, the recombinant gene is integrated into a pT7 vector. In some embodiments, the TIS includes one or more copies of the pT7 vector's original translation initiation nucleotide sequence (OG) (SEQ. ID NO.: 1), recombinant translation initiation nucleotide sequence (UTR) (SEQ. ID NO.: 2), or combinations thereof. In some embodiments, the SS includes one or more polynucleotides encoding Yoch (SEQ. ID NO.: 3), Yngk (SEQ. ID NO.: 4), or combinations thereof. In some embodiments, the recombinant gene includes a structure according to Formula II:

TIS-SSₓ-L-SPₙ (Formula II)

In some embodiments, TIS includes 1-6 copies of OG, UTR, or combinations thereof; SS includes one or more polynucleotides encoding Yoch, Yngk, or combinations thereof; L includes one or more polynucleotides encoding a polypeptide linker; SP includes one or more polynucleotides encoding A5 spidroin (SEQ. ID NO.: 5); x is 1 or more, and n is between about 2 and about 64 repeated gene fragments. In some embodiments, SSₓ-L-SPₙ includes one or more polynucleotides encoding SEQ. ID NO.: 6 (Yoch-L-A5 4mer), SEQ. ID NO.: 7 (Yngk-L-A5 4mer), or combinations thereof.

In some embodiments, preparing a growth medium including the recombinant Bacillus sp. bacteria; producing a recombinant silk protein product in the growth medium via expression of the one or more recombinant genes; and isolating a silk protein product from an extracellular environment surrounding the Bacillus sp. bacteria. In some embodiments, the growth medium includes a minimal media and a concentration of glucose as a carbon source. In some embodiments, the growth medium includes about 1% w/v glucose. In some embodiments, the growth medium includes glutamate, histidine, isoleucine, phenylalanine, proline, tyrosine, lysine, methionine, or combinations thereof. In some embodiments, the growth medium includes 0.3 g/L of glutamate; 0.3 g/L of histidine, isoleucine, phenylalanine, proline, tyrosine, lysine, and methionine; or 0.3 g/L of glutamate, histidine, isoleucine, phenylalanine, proline, tyrosine, lysine, and methionine.

Aspects of the present disclosure are directed to a method of making a recombinant Bacillus sp. bacterium including providing a Bacillus sp. bacterium and inserting one or more recombinant genes to form a recombinant Bacillus sp. bacterium. In some embodiments, the Bacillus sp. bacterium includes B. megaterium. In some embodiments, inserting one or more recombinant genes to form a recombinant Bacillus sp. bacterium includes providing a pT7 vector; inserting the one or more recombinant genes into the pT7 vector to form a recombinant plasmid; and transforming the recombinant plasmid into the Bacillus sp. bacterium.

In some embodiments, the recombinant genes include one or more translational initiation sites (TIS), one or more polynucleotides encoding one or more signal peptides (SS), and one or more gene fragments encoding silk proteins (SP). In some embodiments, the TIS includes one or more copies of the pT7 vector's original translation initiation nucleotide sequence (OG) (SEQ. ID NO.: 1), recombinant translation initiation nucleotide sequence (UTR) (SEQ. ID NO.: 2), or combinations thereof. In some embodiments, the SS includes one or more polynucleotides encoding Yoch (SEQ. ID NO.: 3), Yngk (SEQ. ID NO.: 4), or combinations thereof. In some embodiments, the recombinant gene includes a structure according to Formula II:

TIS-SSₓ-L-SPₙ (Formula II)

In some embodiments, TIS includes 1-6 copies of OG, UTR, or combinations thereof, SS includes one or more polynucleotides encoding Yoch, Yngk, or combinations thereof; L includes one or more polynucleotides encoding a polypeptide linker; SP includes one or more polynucleotides encoding A5 spidroin (SEQ. ID NO.: 5); x is 1 or more, and n is between about 2 and about 64 repeated gene fragments. In some embodiments, SSₓ-L-SPₙ includes one or more polynucleotides encoding SEQ. ID NO.: 6 (Yoch-A5), SEQ. ID NO.: 7 (Yngk-A5), or combinations thereof.

Aspects of the present disclosure are directed to a recombinant B. megaterium bacterium including a plasmid compatible with B. megaterium bacteria and one or more recombinant genes inserted into the plasmid. In some embodiments, the recombinant genes include one or more translational initiation sites (TIS), one or more polynucleotides encoding one or more signal peptides (SS), and one or more gene fragments encoding silk proteins (SP). In some embodiments, the recombinant gene includes a structure according to Formula I:

TISₘ-SSₓ-L-SPₙ (Formula I)

In some embodiments, m is 1-6; SS includes one or more polynucleotides encoding Yoch (SEQ. ID NO.: 3), Yngk (SEQ. ID NO.: 4), or combinations thereof; L includes one or more polynucleotides encoding a polypeptide linker; SP includes one or more polynucleotides encoding A5 spidroin (SEQ. ID NO.: 5); x is one or more, and n is between about 2 and about 64 repeated gene fragments. In some embodiments, the plasmid is a pT7 vector. In some embodiments, the TIS includes one or more copies of the pT7 vector's original translation initiation nucleotide sequence (OG) (SEQ. ID NO.: 1), recombinant translation initiation nucleotide sequence (UTR) (SEQ. ID NO.: 2), or combinations thereof. In some embodiments, SSₓ-L-SPₙ includes one or more polynucleotides encoding SEQ. ID NO.: 6 (Yoch-A5), SEQ. ID NO.: 7 (Yngk-A5), or combinations thereof.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a table showing peptide motifs present in various silk proteins, including spidroins and silk fibroins;

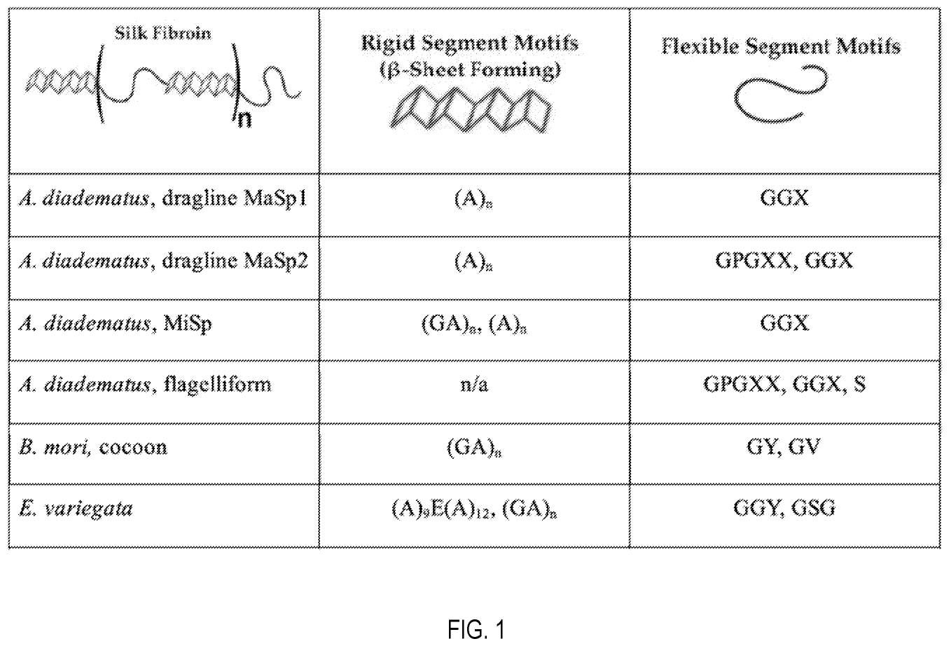

FIG. 2 is a chart of a method of making a recombinant bacterium according to some embodiments of the present disclosure;

FIG. 3 is a chart of a method of making recombinant protein product according to some embodiments of the present disclosure;

FIG. 4 is an image of sodium dodecyl-sulfate polyacrylamide gel electrophoresis (SDS-PAGE) results from nickel-chromatography purification of supernatants of B. megaterium strains expressing recombinant spidroin with Yngk and Yoch signal sequences according to some embodiments of the present disclosure;

FIG. 5 is a graph showing secreted titers of A5 4mer spidroin from B. megaterium strains expressing the spidroin with Yngk and Yoch signal sequences according to some embodiments of the present disclosure;

FIG. 6 is a graph showing secreted titers in Lennox broth (LB) and M9 media of A5 4mer spidroin secreted from B. megaterium strains expressing genes that include altered translational initiation sites (TIS) and polynucleotides encoding Yngk and Yoch signal peptides according to some embodiments of the present disclosure;

FIG. 7 is an image of SDS-PAGE gels from nickel-chromatography purification of recombinant spidroin secreted into the supernatant of B. megaterium strains expressing genes that include altered TIS (UTR6×) according to some embodiments of the present disclosure;

FIG. 8 is a graph showing final OD600 of B. megaterium strain cultures in LB and M9 media according to some embodiments of the present disclosure; and

FIG. 9 is a graph showing secreted titers in LB and M9 media of A5 4mer spidroin from B. megaterium strains expressing the spidroin with Yngk signal sequences according to some embodiments of the present disclosure.

### DETAILED DESCRIPTION

Some embodiments of the present disclosure are directed to recombinant bacteria for the production of a recombinant protein product. In some embodiments, the recombinant bacteria is a modified gram-positive bacterium. In some embodiments, the recombinant bacteria is a modified Bacillus sp. bacteria strain. In some embodiments, the recombinant bacteria is a modified B. megaterium strain. In some embodiments, the B. megaterium strain is strain MS941. B. megaterium MS941 is a derivative of the DSM319 strain in which the only major extracellular protease has been inactivated. In some embodiments, the recombinant bacteria is modified to produce a protein product. In some embodiments, the protein product is a natural protein product, a recombinant protein product, or combinations thereof. In some embodiments, the protein product includes one or more disordered proteins. In some embodiments, the protein product includes silk proteins.

In some embodiments, the recombinant bacteria is modified to express one or more recombinant genes encoding the protein product. In some embodiments, the recombinant bacteria includes one or more plasmids compatible therewith, e.g., compatible with B. megaterium bacteria. In some embodiments, at least one of the plasmids is a recombinant plasmid modified to include the recombinant genes. There are numerous plasmid systems available for use in B. megaterium, many of which have leveraged xylose-inducible promoters to achieve high levels of recombinant protein secretion. In some embodiments, the B. megaterium strain is transformed with a pT7-RNAP vector, e.g., MPS941-pRNAP. In some embodiments, the plasmids include a pT7 vector, pT7-RNAP vector, or combinations thereof. In some embodiments, the recombinant bacteria includes a pT7-RNAP vector and a modified pT7 vector including the one or more recombinant genes. The pT7 vector enables recombinant gene expression using a T7 promoter in the recombinant bacteria when the pT7-RNAP vector is simultaneously present in the strain.

In some embodiments, the recombinant genes include one or more recombinant genes. In some embodiments, the recombinant genes includes one or more polynucleotides encoding one or more recombinant polypeptides. In some embodiments, the recombinant genes include one or more translational initiation sites (TIS), one or more polynucleotides encoding one or more signal peptides (SS), and one or more polynucleotides encoding the protein product. In some embodiments, the polynucleotides include gene fragments encoding silk proteins (SP). In some embodiments, the TIS includes original translation site (OG) of the plasmid into which the recombinant gene is inserted, e.g., the original translation site of the pT7 vector. In some embodiments, the TIS includes OG, a recombinant TIS, or combinations thereof. In some embodiments, TIS form mRNA hairpin structures upon transcription. In some embodiments, the TIS includes SEQ. ID NO.: 1 (OG), SEQ. ID NO.: 2 (UTR), or combinations thereof. In situations where one or more TIS are already present in a bacterium or recombinant plasmid intended for use in that bacterium, it is understood that the recombinant gene can be provided by inserting the SS and SP into position relative to the existing TIS without departing from the intended scope of the present disclosure.

In some embodiments, SS includes one or more polynucleotides encoding Yoch (SEQ. ID NO.: 3), Yngk (SEQ. ID NO.: 4), or combinations thereof. Signal sequences aid in the successful secretion of expressed protein product into an extracellular environment, as will be discussed in greater detail below. In some embodiments, SP includes one or more polynucleotides encoding A5 silk protein sequences. In some embodiments, SP includes one or more polynucleotides encoding SEQ. ID NO.: 5. In some embodiments, SP includes one or more polynucleotides encoding a plurality of repeats of SEQ. ID NO.: 5. In some embodiments, one or more polypeptide linkers are positioned between the signal peptides and the protein product. In some embodiments, the polypeptide linkers include QDHKD, ESVHN, APNNV, ATNTY, DVPTT, or combinations thereof.

In some embodiments, the recombinant gene includes a structure according to Formula I below:

TISₘ-SSₓ-L-SPₙ (Formula I)

In some embodiments, m is 1-12. In some embodiments, m is 1-6. In some embodiments, x is 1 or more. In some embodiments, L includes one or more polynucleotides encoding one or more polypeptide linkers. In some embodiments, n is between about 2 and about 64 repeated gene fragments. In some embodiments, the recombinant gene according to Formula II includes one or more nucleotide additions, deletions, or combinations thereof, so long as the functionality of the individual subunits, e.g., SS, SP, expression thereof, the expressed product, or combinations thereof remain substantially unaffected.

In some embodiments, the recombinant gene includes one copy of OG, one or more polynucleotides encoding one copy of Yoch, and one or more polynucleotides encoding 4 copies of A5. In some embodiments, the recombinant gene includes one copy of OG, one or more polynucleotides encoding one copy of Yngk, and one or more polynucleotides encoding 4 copies of A5. In some embodiments, the recombinant gene includes three copies of OG, one or more polynucleotides encoding one copy of Yoch, and one or more polynucleotides encoding 4 copies of A5. In some embodiments, the recombinant gene includes 6 copies of OG, one or more polynucleotides encoding one copy of Yoch, and one or more polynucleotides encoding 4 copies of A5. In some embodiments, the recombinant gene includes one copy of URT, one or more polynucleotides encoding one copy of Yoch, and one or more polynucleotides encoding 4 copies of A5. In some embodiments, the recombinant gene includes three copies of UTR, one or more polynucleotides encoding one copy of Yoch, and one or more polynucleotides encoding 4 copies of A5. In some embodiments, the recombinant gene includes 6 copies of UTR, one or more polynucleotides encoding one copy of Yoch, and one or more polynucleotides encoding 4 copies of A5. In some embodiments, the protein produced by expressing Formula I includes SEQ. ID NO.: 6, SEQ. ID NO.: 7, or combinations thereof.

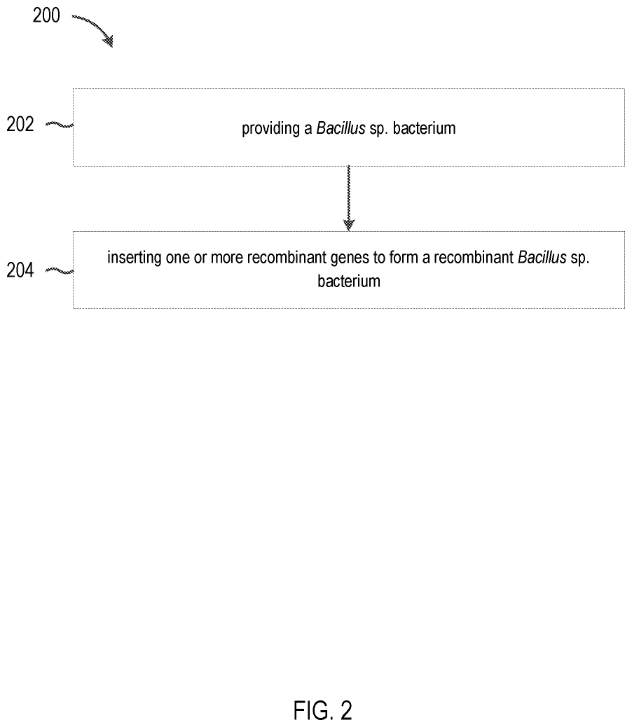

Referring now to FIG. 2, some embodiments of the present disclosure are directed to a method 200 of making a recombinant bacterium. At 202, a bacterium is provided. In some embodiments, the bacterium is a gram-positive bacterium. In some embodiments, the bacterium is a Bacillus sp. bacteria strain. In some embodiments, the bacterium is B. megaterium strain. In some embodiments, the B. megaterium strain is strain MS941.

At 204, one or more recombinant genes are inserted into the bacterium to form a recombinant bacterium. As discussed above, in some embodiments, the recombinant genes include one or more TIS, one or more polynucleotides encoding one or more signal peptides (SS), and polynucleotides encoding the protein product, e.g., gene fragments encoding silk proteins (SP). In some embodiments, the recombinant gene is integrated into a plasmid, e.g., a pT7 vector. In some embodiments, the TIS includes OG, e.g., the original translation site of the pT7 vector. In some embodiments, the TIS includes OG, a recombinant TIS, or combinations thereof. In some embodiments, the TIS includes SEQ. ID NO.: 1 (OG), SEQ. ID NO.: 2 (UTR), or combinations thereof. In some embodiments, SS includes one or more polynucleotides encoding Yoch (SEQ. ID NO.: 3), Yngk (SEQ. ID NO.: 4), or combinations thereof. In some embodiments, SP includes one or more polynucleotides encoding A5 silk protein sequences. In some embodiments, SP includes one or more polynucleotides encoding SEQ. ID NO.: 5. In some embodiments, SP includes one or more polynucleotides encoding a plurality of repeats of SEQ. ID NO.: 5. In some embodiments, the recombinant gene includes a structure according to Formula I above. In some embodiments, the recombinant gene includes a structure according to Formula II below:

TIS-SSₓ-L-SPₙ (Formula II)

In some embodiments, TIS includes 1-6 copies of OG, UTR, or combinations thereof, SS includes one or more polynucleotides encoding Yoch, Yngk, or combinations thereof; L includes one or more polynucleotides encoding one or more polypeptide linkers; SP includes one or more polynucleotides encoding SEQ. ID NO.: 5; x is one or more, and n is between about 2 and about 64 repeated gene fragments. As discussed above, in some embodiments, the recombinant gene according to Formula II includes one or more nucleotide additions, deletions, or combinations thereof, so long as the functionality of the individual subunits, e.g., SS, SP, expression thereof, the expressed product, or combinations thereof remain substantially unaffected. In some embodiments, the protein produced by expressing Formula II includes SEQ. ID NO.: 6, SEQ. ID NO.: 7, or combinations thereof. In some embodiments, inserting 204 includes providing a pT7 vector, inserting the one or more recombinant genes into the pT7 vector to form a recombinant plasmid, and transforming the recombinant plasmid into a bacterium, e.g., B. megaterium.

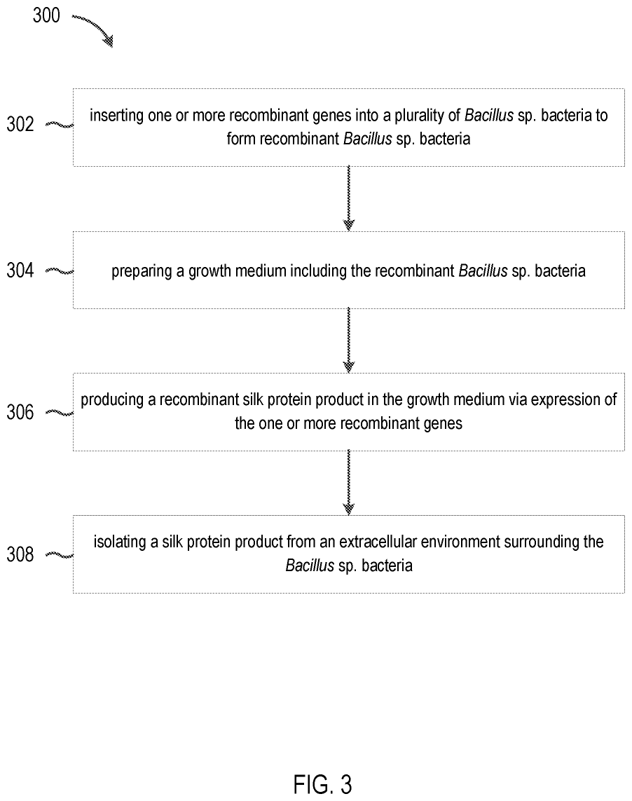

Referring now to FIG. 3, some embodiments of the present disclosure are directed to a method 300 of making recombinant protein product, e.g., silk protein. In some embodiments, at 302, one or more recombinant genes are inserted into a gram-positive bacterium. In some embodiments, the bacterium is Bacillus sp. bacteria. In some embodiments, the recombinant genes are inserted into a plurality of gram-positive bacteria, e.g., Bacillus sp. bacteria. In some embodiments, the plurality of bacteria includes B. megaterium bacteria. As discussed above, in some embodiments, the recombinant genes include one or more TIS, polynucleotides encoding one or more signal peptides (SS), and polynucleotides encoding the protein product, e.g., gene fragments encoding silk proteins (SP). In some embodiments, the recombinant gene is integrated into a plasmid, e.g., a pT7 vector. In some embodiments, the TIS includes OG, e.g., the original translation site of the pT7 vector. In some embodiments, the TIS includes OG, a recombinant TIS, or combinations thereof. In some embodiments, the TIS includes SEQ. ID NO.: 1 (OG), SEQ. ID NO.: 2 (UTR), or combinations thereof. In some embodiments, SS includes one or more polynucleotides encoding Yoch (SEQ. ID NO.: 3), Yngk (SEQ. ID NO.: 4), or combinations thereof. In some embodiments, SP includes one or more polynucleotides encoding A5 silk protein sequences. In some embodiments, SP includes one or more polynucleotides encoding SEQ. ID NO.: 5. In some embodiments, SP includes one or more polynucleotides encoding a plurality of repeats of SEQ. ID NO.: 5. In some embodiments, the recombinant gene includes a structure according to Formula I above. In some embodiments, the recombinant gene includes a structure according to Formula II below:

TIS-SSₓ-L-SPₙ (Formula II)

In some embodiments, TIS includes 1-6 copies of OG, UTR, or combinations thereof; SS includes one or more polynucleotides encoding Yoch, Yngk, or combinations thereof; L includes one or more polynucleotides encoding one or more polypeptide linkers; SP includes one or more polynucleotides encoding SEQ. ID NO.: 5; x is one or more, and n is between about 2 and about 64 repeated gene fragments. As discussed above, in some embodiments, the recombinant gene according to Formula II includes one or more nucleotide additions, deletions, or combinations thereof, so long as the functionality of the individual subunits, e.g., SS, SP, expression thereof, the expressed product, or combinations thereof remain substantially unaffected. In some embodiments, the protein produced by expressing Formula II includes SEQ. ID NO.: 6, SEQ. ID NO.: 7, or combinations thereof.

At 304, a growth medium is prepared. In some embodiments, the growth medium includes and is configured to facilitate proliferation of the modified bacteria from step 302, e.g., recombinant B. megaterium bacteria, as well as the expression of the recombinant genes by those recombinant bacteria. In some embodiments, the growth medium includes a minimal media. In some embodiments, the minimal media includes modified M9 media. The use of minimal media has several advantages, including that it can be considerably more cost effective at the industrial scale. Additionally, its chemical composition is more precisely defined versus, e.g., complex media derived from peptones, enabling more reproducible outcomes and enhanced analytical analysis.

In some embodiments, the growth medium includes a concentration of one or more amino acids including glutamate, histidine, isoleucine, phenylalanine, proline, tyrosine, lysine, and methionine. In some embodiments, the concentration of amino acids in the growth medium is about 0.3 g/L. In some embodiments, the growth medium includes 0.3 g/L of glutamate. In some embodiments, the growth medium includes 0.3 g/L of histidine, isoleucine, phenylalanine, proline, tyrosine, lysine, and methionine. In some embodiments, the growth medium includes 0.3 g/L of glutamate, histidine, isoleucine, phenylalanine, proline, tyrosine, lysine, and methionine. In some embodiments, the growth medium is supplemented with one or more trace salts. In some embodiments, the trace salts are provided at a concentration below about 50 mM. In some embodiments, the trace salts include Na₂HPO₄, KH₂PO₄, NH₄Cl, NaCl, CaCl₂, MgSO₄, FeSO₄, MnSO₄, or combinations thereof. In some embodiments, the trace salts include 48 mM Na₂HPO₄, 22 mM KH₂PO₄, 18.8 mM NH₄Cl, 8.6 mM NaCl, 0.1 mM CaCl₂), 2 mM MgSO₄, 36 μM FeSO₄, and 4.14 μM MnSO₄. In some embodiments, the growth medium includes one or more carbon sources. In some embodiments, the growth medium includes a concentration of glucose as a carbon source. In some embodiments, the growth medium includes about 1% w/v glucose.

Still referring to FIG. 3, at 306, a recombinant silk protein product is produced in the growth medium via expression of the one or more recombinant genes. Gram-positive hosts can offer advantages for recombinant silk production over gram-negative hosts due to their strong secretion capabilities, avoidance of intracellular inclusion bodies, and their generally recognized as safe (GRAS) status by the United States Food and Drug Administration. Furthermore, intracellularly produced proteins, e.g., spidroins, common in engineered E. coli strains for production of spidroin products, can exert product toxicity effects and exacerbate unfavorable stress mechanisms within the host. Thus, the protein product produced at step 306 can be secreted into the growth medium for collection, avoiding cellular toxicity, and advantageously enabling continuous production and harvesting of the product by eliminating the need to lyse the cells to obtain that product. At 308, a protein product is isolated from an extracellular environment surrounding the recombinant bacteria, i.e., after secretion by the recombinant bacteria. In some embodiments, the protein product is isolated 308 from the growth medium.

Unlike gram-negative bacteria, secretion of recombinant proteins into the culture media by a gram-positive species can be achieved with the Sec secretion pathway, which can be favorable to all other secretion pathways including the TaT or Type 1-6 pathways. Indeed, secretory production in the gram-negative S. typhimurium using a type III secretion pathway was found to have severe shortcomings. Achieving secretion used the implementation of a multifaceted genetic circuit that modulated chaperone proteins, cellular secretion needles, and protease activity. Utilization of the Sec pathway involves the addition of a short N-terminal tag on the protein of interest and additional genetic circuits are unnecessary. This N-terminal sequence is known as the signal peptide, or signal sequence, and a given gram-positive species can have over 180 endogenous sequences that can be leveraged for synthetic biology applications. Further, secretion of a protein product from the host cell using the Sec system in gram-positive species passes through only one membrane and the action of only one transport protein, versus two membranes at least two transporters for gram-negative secretion pathways. Finally, secretion through the Sec pathways includes the cleavage of the signal peptide from the protein product during passage through the cell membrane, enabling the purification of an unaltered recombinant protein from a culture supernatant.

### EXAMPLES

A plurality of exemplary recombinant bacterial strains consistent with the embodiments discussed above were prepared and analyzed. In some exemplary embodiments, E. coli and B. megaterium MS941 parent strain were both grown in LB (Lennox broth, Sigma Aldrich, St. Louis, MO). For cloning and plasmid preparation, E. coli was cultured in shake flasks at 37 °C with shaking at 225 rpm and supplemented with 100 μg/ml ampicillin. In exemplary embodiments, B. megaterium was grown in M9 minimal media supplemented with trace minerals (48 mM M Na₂HPO₄, 22 mM KH₂PO₄, 18.8 mM NH₄Cl, 8.6 mM NaCl, 0.1 mM CaCl₂, 2 mM MgSO₄, 36 μM Fe SO₄, 4.14 μM MnSO₄) and 1% w/v glucose as a carbon source. In some exemplary embodiments, B. megaterium was grown in the aforementioned M9 media supplemented with 0.3 g/L of glutamate (one amino acid); 0.3 g/L of histidine, isoleucine, phenylalanine, proline, tyrosine, lysine, and methionine (seven amino acids); or 0.3 g/L of glutamate, histidine, isoleucine, phenylalanine, proline, tyrosine, lysine, and methionine (eight amino acids). Some exemplary cultures of B. megaterium grown in M9 media with 0.3 g/L of the eight amino acids were supplemented with 10-, 30-, or 90-mM sodium acetate at the time of recombinant gene induction. Cultures of B. megaterium containing recombinant gene cassettes were supplemented with tetracycline at 40 μg/ml and chloramphenicol at 25 μg/ml when grown in LB media, or 20 μg/ml tetracycline and 10 μg/ml chloramphenicol when grown in M9 media and M9 media variants.

To prepare B. megaterium strains for secreting recombinant silk protein consistent with embodiments of the present disclosure, a variant of the A5 4mer recombinant spidroin sequence (SEQ. ID NO.: 8) was prepared. The A5 4mer gene was designed based on amino acid motifs present on MaSp2 spider dragline silk. However, there are slight differences in the primary sequence of the version produced in B. megaterium (SEQ. ID NO.: 8) versus that of E. coli. The construct produced in E. coli included an 18-residue spacer between the histidine tag the start of the A5 4mer spidroin primary sequence of gene fragment repeats, which B. megaterium lacked. Additionally, the final four residual amino acids (TSGS) found in the E. coli A5 4mer version were removed from the B. megaterium construct. Furthermore, the A5 4mer produced in B. megaterium had a histidine tag that was shortened from 10 tandem histidine residues to 6. Moreover, the histidine tag on the construct produced in B. megaterium was moved from the N-terminus to the C-terminus to help facilitate Sec production. The A5 4mer genes were initially cloned into five variants of a pT7 vector backbone using the SalI and SpeI restriction sites.

The five variants of pT7 vector used for these exemplary embodiments each contain polynucleotides encoding a signal peptide (SS) and linker sequence prior to the start of the recombinant silk gene (SP) to enable secretion of the construct. Exemplary embodiments prepared included pT7-aamy-A5, pT7-LipA-A5, pT7-Nprm-A5, pT7-Yoch-A5, and pT7-Yngk-A5. For creation of strains with multiple tandem translation initiation sites (TISs) or untranslated regions designed to form mRNA hairpin structures also consistent with embodiments described above, gene fragments including 3× and 6× copies of the original translation site identified in SEQ. ID NO.: 1 (named OG3× and OG6× respectively) and 1×, 3×, and 6× copies of the recombinant translation site identified in SEQ. ID NO.: 2 (named UTR1×, UTR3×, and UTR6× respectively) were prepared and cloned in the pT7-Yoch-A5 construct using the BspTI and NheI restriction sites. Each TIS contained a copy of the T7 promoter region found on the pT7 vector backbone upstream of the translation initiation sequence to facilitate ease of cloning. A strain containing the A5 4mer gene but lacking a signal sequence and linker (no secretion strain) was created by inserting an A5 4mer gene in a purchased pBluescript II SK(+) vector backbone with NheI replacing the original SalI N-terminal restriction site. The construct was then cloned into a pT7-Yoch-A5 backbone using the NheI and SalI sites to remove the Yoch signal sequence and linker and yield pT7-A5.

Transformations of vectors into B. megaterium were performed. The pT7-RNAP vector was first transformed into B. megaterium to form a parent strain. All pT7 vector variants containing an A5 4mer spidroin sequence were then transformed into this parent strain for recombinant silk expression and secretion experiments. All colonies of B. megaterium used for silk expression were sequenced to confirm proper transformation of the pT7 constructs.

Recombinant silk expressions were carried out by first inoculating 25 ml cultures with a single plated colony and incubating overnight at 37 °C with shaking at 225 rpm. For expression in LB media, overnight cultures were grown in LB media. For expression in exemplary embodiments with M9 media and M9 variants, overnight cultures were grown in either M9 or LB media depending on the specific strain's ability to grow overnight in M9 media. 300 ml expression cultures (placed in 1-liter flasks) were inoculated with 6 ml of an overnight culture and grown at 37 °C with shaking at 225 rpm. Cultures were induced for spidroin expression via the addition of 0.5% or 1.5% w/v xylose when the OD600 was between 0.275-0.325. Cultures were either kept at 37 °C or shifted to 30 °C or 20 °C upon induction and total expression times were 21 hours.

Expression cultures were centrifuged at 7,500 rcf for 30 minutes to separate the culture supernatant from the cell pellet. For analysis on intracellular production, cell pellets were first put through a freeze thaw cycle before being resuspended in 2 ml/per gram cell pellet of B-PER II Bacterial Protein Extraction Reagent (Thermo Fisher Scientific, Waltham, MA) supplemented with 20 mg/ml lysozyme, 1 mg/ml DNaseI, and 1 mM PMSF. The mixture was then incubated at 37 °C with shaking at 225 rpm for two hours before being vortexed for 60 seconds. The cell lysate was then centrifuged at 7,500 rcf for 30 minutes to pellet the insoluble debris. Both the soluble and insoluble fractions of the lysates were put through nickel affinity chromatography and sodium dodecyl-sulfate polyacrylamide gel electrophoresis (SDS-PAGE) to identify if intracellular silk production had occurred. For analysis on secreted production, culture supernatants were first concentrated from 300 ml to 25 ml using Amicon centrifugal filters (Sigma Aldrich, St. Louis, MO). The concentrated supernatants were then put through nickel affinity chromatography and SDS PAGE. Elution fractions after chromatography (for both intracellular and secreted analyses) were concentrated up to 5-fold using Amicon centrifugation filters before SDS PAGE analysis to confirm the absence of recombinant protein in cases where no protein bands were seen on unconcentrated elution fractions.

The titers of secreted A5 4mer spidroin were calculated using the Pierce BCA Protein Assay Kit on the elution fractions of chromatography (Thermo Fisher Scientific, Waltham, MA). It should be noted that while the translated signal sequence from each pT7 vector construct is cleaved during secretion, 5-amino acid long linkers remained at the N-terminal end of the secreted A5 4mer protein. Since the secreted A5 4mer proteins from the Yoch and Yngk signal sequences had slightly different linker sequences that are included in the final secreted construct, a standard curve for each was created in order to accurately calculate concentrations using the BCA assay. Secreted A5 4mer protein that was created from both the Yoch and Yngk strains was purified and dialyzed into water using SnakeSkin Dialysis Tubing (Thermo Fisher Scientific, Waltham, MA) before being lyophilized. The lyophilized proteins were weighed and dissolved in the same elution buffer used in the chromatography procedure to make standard solutions ranging from 4 to 0.0625 mg/ml. A trendline equation for each construct with R2>0.99 was used for subsequent titer calculations.

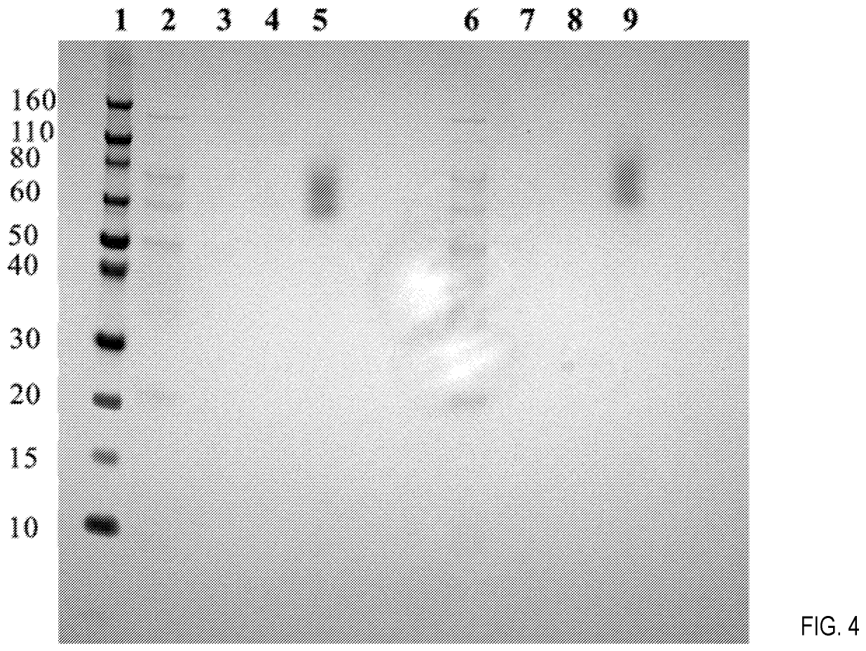

Referring now to FIG. 4, in preliminary experiments performed in LB media at 37 °C in shake flasks, the Yngk and Yoch signal sequences enabled successful secretion of the silk protein (strains pT7-Yoch-A5, and pT7-Yngk-A5). For all signal sequences tested no concurrent intracellular production was observed, and any silk protein produced in the host was secreted. It was observed through SDS-PAGE that the version of the A5 4mer produced in B. megaterium exhibited different behavior versus that of E. coli. The version produced in E. coli showed an apparent molecular weight on SDS PAGE of 38 kDa with respect to the protein ladder, while the B. megaterium version can be seen at approximately 60 kDa. This increase in apparent molecular weight is not due to the presence of a signal peptide, as that is cleaved from the silk protein during Sec secretion. Rather, without wishing to be bound by theory, this difference in apparent molecular weight (increase in aberrant mobility) indicates an increased level of intrinsic disorder in the B. megaterium construct. Moreover, the B. megaterium A5 4mer shows much weaker staining with Coomassie blue. Again, without wishing to be bound by theory, these differences are likely due to the slight changes in primary sequence between the B. megaterium and the E. coli A5 4mer primary sequence. Specifically, truncation of a histidine tag has been found to increase structural disorder within proteins. Likewise, Coomassie blue interacts primarily with arginine, tyrosine, lysine, and histidine residues, and the histidine tag on the A5 4mer sequence is the portion of the peptide that contains such amino acids.

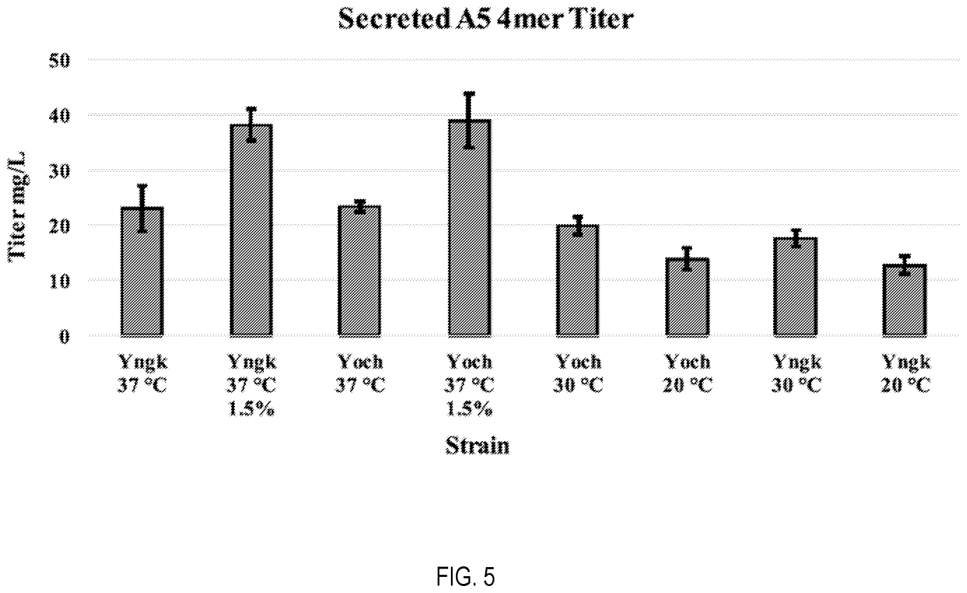

Referring now to FIG. 5, the Yngk and Yoch strains were tested for their A5 4mer secretion levels at various temperatures and xylose (inducer) concentrations in LB media. The secreted titer for both strains was found to be 23 mg/L at 37 °C with a 0.5% w/v xylose induction. Lowering the expression temperature to 30 °C in conjunction with a 0.5% w/v xylose induction was found to lower titers to 17 mg/L and 20 mg/L for Yngk and Yoch, respectively. Likewise, lowering the expression temperature to 20 °C in conjunction with a 0.5% w/v xylose induction was found to further lower titers to 12 mg/L and 13 mg/L for Yngk and Yoch, respectively. However, if expressions were performed at 37 °C with a stronger xylose induction (1.5% w/v) titers were observed to increase to 38 mg/L for both strains, representing a 65% increase versus 0.5% w/v xylose.

Notably, the secreted titers for strains Yngk and Yoch remain practically identical across the conditions tested. This is unexpected as variation in the signal sequence can result in substantial changes in the secreted titer of a recombinant protein, including order of magnitude differences. The A5 4mer is a small (approximately 16 kDa) and disordered recombinant protein, and the Sec secretion machinery is particularly efficient at secreting small proteins in an unfolded state. This, combined with the observation that none of the strains identified in the embodiments of the present disclosure exhibit concurrent intracellular spidroin production, indicates that secretion may not be a bottleneck for small, disordered spidroin production in B. megaterium. Rather, other bottlenecks may be involved such as metabolic burden or product toxicity. To test this, an additional expression strain was created in which the Yoch signal sequence and linker sequence were removed from the expression vector. Therefore, this non-secreting strain should produce an identical version of the A5 4mer silk protein but retain the construct intracellularly. However, production of the A5 4mer was not detected for the non-secreting strain, despite confirmation that the strain contained the recombinant vector prior to expression in LB media at 37 °C with 1.5% w/v xylose, thus demonstrating the potential benefits of secretion for the production of toxic, disordered recombinant proteins.

Translation efficiency depends on multiple factors, including the speed of translation elongation and the rate at which translation is initiated. Translation initiation specifically refers to the binding of a ribosome to mRNA at the ribosome binding site (RBS) followed by the scanning of the ribosome along the mRNA's untranslated region to identify a start codon and initiate translation. The RBS, untranslated region, and start codon can together be referred to as the translation initiation site or TIS. This is the first step in the translation process and is of interest for recombinant protein production in gram-positive bacteria, as multiple TISs on a gene can all be active during translation in gram-positive bacteria, however, only one TIS can be active for a given gene in the gram-negative E. coli.

In an effort to increase secreted spidroin titer and identify potential bottlenecks, the Yoch strain in conjunction with expressions in LB media at 37 °C with 1.5% w/v xylose were chosen as starting points. Five new strains were created from a pT7-Yoch-A5 backbone to understand if polymerized TISs (multiple tandem TISs) and an alternative TIS designed to form a hairpin structure could increase titers of secreted silk. Two of the strains, OG3× and OG6× identified above, contained the original translation initiation site present on the expression vector backbone (inclusive of the ribosome binding site and start codon) that had been polymerized in tandem from one copy to three and six copies, respectively. A different strain was created (UTR1× identified above) in which a single copy of an alternative translation initiation site (SEQ. ID NO.: 2) was swapped for the original. This alternative translation initiation site (UTR) was intended to form a hairpin structure with the ribosome binding site contained on the loop of the hairpin and the start codon present directly after the hairpin stem. Two final strains were created, UTR3× and UTR6× identified above, in which the translation initiation site present on UTR1× was polymerized in tandem (inclusive of the ribosome binding site and start codon) to three and six copies, respectively. These plasmids were used to create five new strains (OG3×, OG6×, UTR1×, UTR3×, and UTR6×) and all contained a single Yoch signal sequence and Yoch linker sequence gene after the translation initiation site(s), and a single copy of the A5 4mer recombinant silk gene.

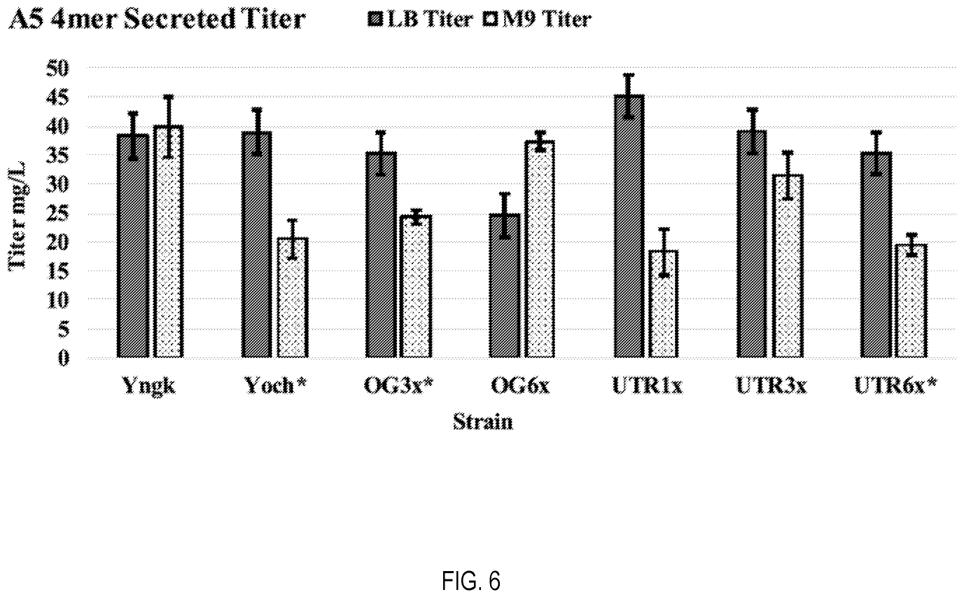

Referring now to FIG. 6, expressions were carried for 21 hours at 37 °C in LB media with 1.5% w/v xylose supplemented for recombinant gene induction. Results showed that secreted titers with the altered translation initiation sites varied from a low of 24.5±3.8 mg/L with the OG6× strain to a high of 45.1±3.7 mg/L with the UTR1× strain. The titer of 45.1±3.7 mg/L for strain UTR1× is approximately 15% higher than that achieved with the Yoch parent strain. It was notable that none of the five strains with altered TISs produced any intracellular silk, all production was found in the secreted fraction.

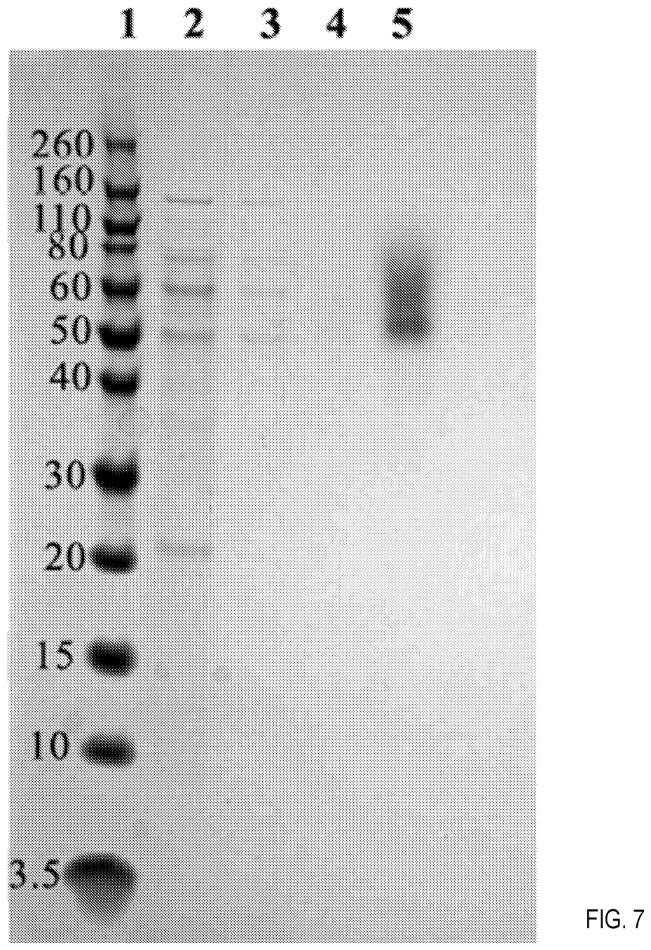

Referring now to FIG. 7, SDS PAGEs showed that purified silk from all TIS-altered strains was found to appear at the same apparent molecular weight (approximately 60 kDa) as the construct produced in the parent Yoch strain. Strains OG3×, OG6×, UTR3×, and UTR6× contain multiple start codons upstream of the Yoch signal sequence, meaning that there are N-terminal amino acid sequences prior to the Yoch signal peptide on the newly translated (but not secreted) A5 4mer preprotein. Thus, it appears that the added N-terminal sequences did not affect ability of the host to secrete the construct (no intracellular production detected) or cleave the signal peptide from the silk protein during Sec secretion (proper apparent weight of SDS-PAGE).

Referring again to FIG. 6, exemplary embodiments of the present disclosure using M9 minimal media were prepared. Minimal media expressions were carried out in M9 minimal media with 1% w/v glucose for strains Yngk, Yoch, OG3×, OG6×, UTR1×, UTR3×, and UTR6×. All expressions were carried out for 21 hours at 37 °C with 1.5% w/v xylose supplemented for recombinant gene induction. Titers in M9 media for the Yoch, OG3×, UTR1×, and UTR6× strains were approximately 20 mg/L, representing a decrease of 43-55% versus LB media (FIG. 3). The titers for strains Yngk and UTR3× in M9 remained approximately equal to those in LB, with the secreted titer of 39.8±5.2 mg/L in Yngk representing the highest titer in M9 for all strains tested. The OG6× strain was the only strain to show a non-negligible increase in titer of 24±3.8 to 37±1.5 mg/L when transitioning from LB to M9. For the OG3× to the OG6× strains, titers increased in M9 media by 54% (+13 mg/L). Strain Yngk (having a single TIS) yielded the highest titer out of all strains in M9 media. No concurrent intracellular production was observed for any strains in M9 media.

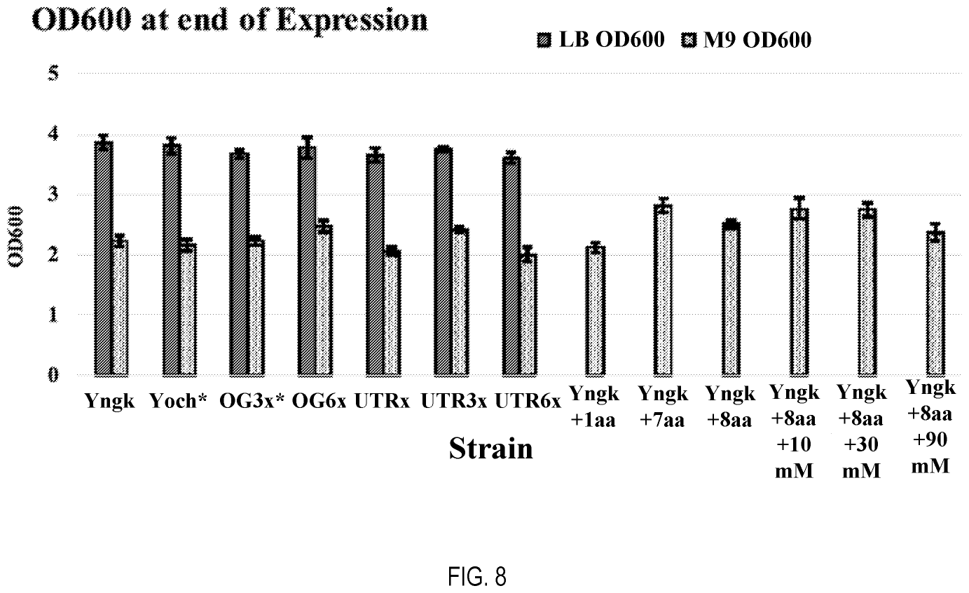

Referring now to FIG. 8, the strains producing spidroin titers in M9 that are equal to or exceeding those measured in LB did so while accumulating substantially less biomass in M9. The OD600 at the end of expressions in LB or M9 was consistent across all strains and was only dependent on the media used. The final OD600 in LB ranged from 3.6 to 3.86, and from 2-2.47 in M9 media. It was observed that certain strains were unable to grow when overnight M9 cultures were inoculated with a single plated colony (despite these strains growing adequately in overnight LB cultures), namely Yoch, OG3×, and UTR6×. Therefore, expression cultures in M9 media for these strains were inoculated from LB overnight cultures, however, an extended lag phase in the expression cultures was observed. Expression cultures in M9 media for strains that grew well overnight in M9 reached the OD600 target for induction (0.275-0.325) within 2-3 hours after inoculation. However, the Yoch, OG3×, and UTR6× M9 expression cultures took approximately 5-6 hours to reach the target OD600 for induction, despite being inoculated from dense LB overnight cultures. These differences in growth after inoculation among the strains did not yield sizable differences in the OD600 at the end of expressions in M9 media (see FIG. 8). Comparing the lagged growth of Yoch to the robust growth of Yngk in M9 media, without wishing to be bound by theory, it appears the signal sequence and linker sequence affect performance of a strain across media conditions (both the Yoch strain and the Yngk strain have the same TIS).

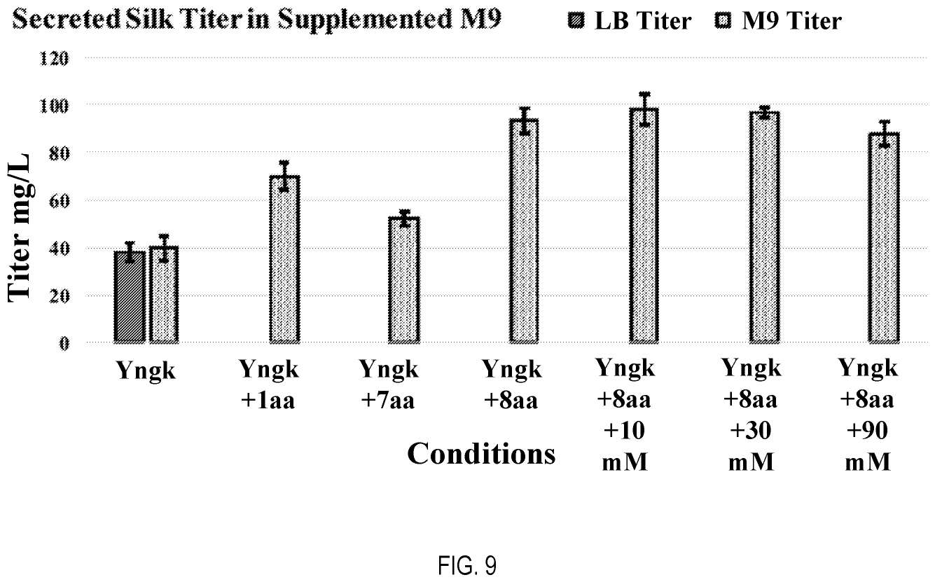

Referring now to FIG. 9, the ability of the Yngk strain to maintain a titer of 40 mg/L in LB and M9 is significant when compared to a previously tested SoluBL21-pLysS E. coli strain, for which an 82% decrease in A5 4mer production was observed when transitioning from LB to M9. It appears that the Yngk B. megaterium strain is more tolerant to glucose as a carbon source during recombinant silk production. Expressions were performed with the Yngk strain in M9 media using the same amino acid supplementation protocol that can relieve metabolic bottlenecks in E. coli. When 0.3 g/L of glutamate was applied to the cultures (+1aa), the secreted titer increased by 76% from 39.8±5.2 mg/L to 70±5.7 mg/L when compared to LB or unaltered M9 media. Cultures lacking glutamate, but supplemented with 0.3 g/L of histidine, isoleucine, phenylalanine, proline, tyrosine, lysine, and methionine (+7aa) yielded a titer of 52.3±3.0 mg/L, a 31% increase in titer compared to LB or unaltered M9. When 0.3 g/L of glutamate and 0.3 g/L of the seven amino acids were supplemented together (+8aa), the Yngk strain exhibited at 135% increase in A5 4mer production versus LB or unaltered M9, with a titer of 93.4±5.4 mg/L. This represents the highest titer achieved for any B. megaterium strain or expression conditions tested and is approximately equal to the production levels in LB media for the E. coli pLysS and SoluBL21 parent strains that were used to create SoluBL21-pLysS strains for maximizing spidroin titers.

However, in M9 media supplemented with 0.3 g/L of eight amino acids (+8aa), the SoluBL21-pLysS strain still yielded A5 4mer titers 57% lower than in LB media. This contrasts with the B. megaterium system in which the M9 with eight amino acids outperforms LB media by 145%. Without wishing to be bound by theory, a possible factor is the repression of the xylA promoter by up to 13-fold in B. megaterium when glucose is present in the media. The xylA promoter controls the production of the T7 promoter, with the T7 promoter controlling the expression of the A5 4mer gene. Downregulation of the promoter strength can be advantageous for producing disordered recombinant silk proteins in gram-positive species. The increased biomass during expression for B. megaterium in LB media versus M9 media plus eight amino acids is also noteworthy when considering the higher titers for the latter. It is possible that the amino-acid rich, but carbohydrate depleted, LB media preferentially directs metabolic fluxes to biomass as opposed to silk synthesis, whereas in M9 minimal media the abundance of glucose as a carbon and energy source enables a preferential metabolic flux of supplemented amino acids towards silk synthesis.

Still referring to FIGS. 8 and 9, expression cultures of the Yngk strain in M9 media with 0.3 g/L of eight amino acids (+8aa) were supplemented with acetate (10, 30, or 90 mM of exogenous acetate added at time of induction). Results for strain Yngk showed that the previous titer of 93 mg/L in M9 with eight amino acids was maintained across all acetate concentrations tested, with titers during acetate supplementation ranging from 88-98 mg/L (see FIG. 9). This contrasts with E. coli SoluBL21-pLysS in which a 52, 57, and 85% decrease in titer was observed at 10-, 30-, and 90-mM acetate, respectively. Likewise, the final OD600 of strain Yngk at the various acetate levels was approximately equal to that in the baseline media, ranging from 2.4-2.7 (see FIG. 8). In contrast, the SoluBL21-pLysS strain showed reductions in final OD600 of 28, 28, and 39% at 10-, 30-, and 90-mM acetate, respectively. These results suggest secretion can break negative metabolic feedback loops within a microbial host by, e.g., mitigating the product toxicity of disordered recombinant spidroins, which further can benefit scaled production involving high biomass levels. Moreover, secretion of silk proteins is a useful tool when minimal media and a glucose feedstock are desired for a given bioprocess design.

Systems and methods of the present disclosure advantageously utilize recombinant B. megaterium bacteria to express and secrete disordered protein products, such as spidroins. The gram-positive B. megaterium bacteria were modified with particular combinations of translation initiation sites and polynucleotides encoding signal peptides to facilitate expression of recombinant gene fragments encoding disordered silk proteins at concentrations comparable to SoluBL21-pLysS E. coli strains specifically designed for maximizing silk protein titers. The ability of the modified B. megaterium bacteria to not only generate the desired product but also secrete that product into the growth media was found to mitigate the product toxicity of the A5 4mer protein product and promote favorable outcomes in a glucose-based media. Targeted amino acid supplementation in minimal growth media for the modified bacteria relieved bottlenecks and enabled secreted recombinant silk titers of 98.2±6.5 mg/L, a 133% increase in titer versus LB or unaltered M9 media, representing the highest reported secreted silk titers for a bacterial host.

The B. megaterium secretion host systems consistent with embodiments of the present disclosure can lower cost of silk protein production through reduced purification complexity, time, and associated capital expenditures. In addition, secretion host systems eliminate the need for cell lysis and improve the economics of industrial silk production. Thus, the systems and methods of the present disclosure facilitate a continuous bioprocess, eliminating production downtimes, product variation, decreasing inventory needs and cost of goods, increasing scalability, and reducing operating costs and equipment size. Further, a bacterial system such as the one described in the present disclosure has several advantages over alternatives such as yeast and insect cells, including less concern over product-destroying proteases, faster growth and production times, and simpler genetic and phenotypic manipulation of the cells.

Silk proteins, and the present methods of producing them, have several advantages over current and potential alternatives. In contrast to silk proteins, most currently available customizable and high-performance polymers and materials are nonbiodegradable, and most lack biocompatibility, thus giving silk an advantage over current widely used polymeric materials. Previous methods of acquiring silk, such as sericulture (cultivating silkworms and harvesting their cocoons), are time-intensive and face severe limitations when it comes to scalability and material diversity (silkworms need specific living conditions and only produce one type of silk at a finite rate). Previous recombinant spidroin production methods can produce many different variations of silk protein, but suffer from very low yields and batch production limitations, contributing to silk's biggest disadvantage: high product prices when compared to commonly used polymers.

Thus, the present disclosure can advantageously produce recombinant spidroin, which have diverse uses as biocompatible and biodegradable materials for biomedical applications, such as drug delivery, implants, and tissue engineering; consumer products, such as cosmetics, sustainable clothing, fabrics, and textiles; and food packaging, such as coatings for food preservation. Additional background relevant to embodiments of the present disclosure can be found in WO 2022/177949, the contents of which are incorporated by reference herein in its entirety.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A method of making recombinant silk protein, comprising:
inserting one or more recombinant genes into a plurality of Bacillus sp. bacteria to form recombinant Bacillus sp. bacteria, wherein the recombinant genes include:
one or more translational initiation sites (TIS);
one or more polynucleotides encoding one or more signal peptides (SS); and
one or more gene fragments encoding silk proteins (SP),
preparing a growth medium including the recombinant Bacillus sp. bacteria;
producing a recombinant silk protein product in the growth medium via expression of the one or more recombinant genes; and
isolating a silk protein product from an extracellular environment surrounding the Bacillus sp. bacteria,
wherein the plurality of Bacillus sp. bacteria include B. megaterium bacteria.

2. The method according to claim 1, wherein the recombinant gene is integrated into a pT7 vector.

3. The method according to claim 2, wherein the TIS includes one or more copies of the pT7 vector's original translation initiation nucleotide sequence (OG) (SEQ. ID NO.: 1), recombinant translation initiation nucleotide sequence (UTR) (SEQ. ID NO.: 2), or combinations thereof.

4. The method according to claim 1, wherein the SS includes one or more polynucleotides encoding Yoch (SEQ. ID NO.: 3), Yngk (SEQ. ID NO.: 4), or combinations thereof.

5. The method according to claim 1, wherein the recombinant gene includes a structure according to Formula II:
TIS-SSₓ-L-SPₙ (Formula II)
wherein TIS includes 1-6 copies of OG, UTR, or combinations thereof; SS includes one or more polynucleotides encoding Yoch, Yngk, or combinations thereof; L includes one or more polynucleotides encoding a polypeptide linker; SP includes one or more polynucleotides encoding A5 spidroin (SEQ. ID NO.: 5); x is 1 or more, and n is between about 2 and about 64 repeated gene fragments.

6. The method according to claim 5, wherein SSₓ-L-SPₙ includes one or more polynucleotides encoding SEQ. ID NO.: 6, SEQ. ID NO.: 7, or combinations thereof.

7. The method according to claim 1, wherein the growth medium includes a minimal media and a concentration of glucose as a carbon source.

8. The method according to claim 7, wherein the growth medium includes about 1% w/v glucose.

9. The method according to claim 7, wherein the growth medium includes glutamate, histidine, isoleucine, phenylalanine, proline, tyrosine, lysine, methionine, or combinations thereof.

10. The method according to claim 9, wherein the growth medium includes:
0.3 g/L of glutamate;
0.3 g/L of histidine, isoleucine, phenylalanine, proline, tyrosine, lysine, and methionine; or
0.3 g/L of glutamate, histidine, isoleucine, phenylalanine, proline, tyrosine, lysine, and methionine.

11. A method of making a recombinant Bacillus sp. bacterium, comprising:
providing a Bacillus sp. bacterium;
inserting one or more recombinant genes to form a recombinant Bacillus sp. bacterium, wherein the recombinant genes include:
one or more translational initiation sites (TIS);
one or more polynucleotides encoding one or more signal peptides (SS); and
one or more gene fragments encoding silk proteins (SP),
wherein the Bacillus sp. bacterium includes B. megaterium.

12. The method according to claim 11, wherein inserting one or more recombinant genes to form a recombinant Bacillus sp. bacterium includes:
providing a pT7 vector;
inserting the one or more recombinant genes into the pT7 vector to form a recombinant plasmid; and
transforming the recombinant plasmid into the Bacillus sp. bacterium.

13. The method according to claim 12, wherein the TIS includes one or more copies of the pT7 vector's original translation initiation nucleotide sequence (OG) (SEQ. ID NO.: 1), recombinant translation initiation nucleotide sequence (UTR) (SEQ. ID NO.: 2), or combinations thereof.

14. The method according to claim 11, wherein the SS includes one or more polynucleotides encoding Yoch (SEQ. ID NO.: 3), Yngk (SEQ. ID NO.: 4), or combinations thereof.

15. The method according to claim 11, wherein the recombinant gene includes a structure according to Formula II:
TIS-SSₓ-L-SPₙ (Formula II)
wherein TIS includes 1-6 copies of OG, UTR, or combinations thereof; SS includes one or more polynucleotides encoding Yoch, Yngk, or combinations thereof; L includes one or more polynucleotides encoding a polypeptide linker; SP includes one or more polynucleotides encoding A5 spidroin (SEQ. ID NO.: 5); x is 1 or more, and n is between about 2 and about 64 repeated gene fragments.

16. The method according to claim 15, wherein SSₓ-L-SPₙ includes one or more polynucleotides encoding SEQ. ID NO.: 6, SEQ. ID NO.: 7, or combinations thereof.

17. A recombinant B. megaterium bacterium comprising:
a plasmid compatible with B. megaterium bacteria; and
one or more recombinant genes inserted into the plasmid, wherein the recombinant genes include:
one or more translational initiation sites (TIS);
one or more polynucleotides encoding one or more signal peptides (SS); and
one or more gene fragments encoding silk proteins (SP),
wherein the recombinant gene includes a structure according to Formula I:
TISₘ-SSₓ-L-SPₙ (Formula I)
and wherein
m is 1-6;
SS includes one or more polynucleotides encoding Yoch (SEQ. ID NO.: 3), Yngk (SEQ. ID NO.: 4), or combinations thereof;
L includes one or more polynucleotides encoding a polypeptide linker;
SP includes one or more polynucleotides encoding A5 spidroin (SEQ. ID NO.: 5);
x is one or more, and
n is between about 2 and about 64 repeated gene fragments.

18. The recombinant B. megaterium bacterium according to claim 17, wherein the plasmid is a pT7 vector.

19. The recombinant B. megaterium bacterium according to claim 18, wherein the TIS includes one or more copies of the pT7 vector's original translation initiation nucleotide sequence (OG) (SEQ. ID NO.: 1), recombinant translation initiation nucleotide sequence (UTR) (SEQ. ID NO.: 2), or combinations thereof.

20. The recombinant B. megaterium bacterium according to claim 19, wherein SSₓ-L-SPₙ includes one or more polynucleotides encoding SEQ. ID NO.: 6, SEQ. ID NO.: 7, or combinations thereof.
